(19) United States
(12) Patent Application Publication
(10) Pub. No.: US 2015/0286788 A1
(43) Pub. Date: Oct. 8, 2015

(54) Operating Room Management System with Smart Chart for Anesthesia Monitoring

(71) Applicant: Harold Arkoff, Sudbury, MA (US)

(72) Inventor: Harold Arkoff, Sudbury, MA (US)

(21) Appl. No.: 14/682,092

(22) Filed: Apr. 8, 2015

Related U.S. Application Data
(60) Provisional application No. 61/976,625, filed on Apr. 8, 2014.

(51) Int. Cl. G06F 19/00; A61B 5/021; A61B 5/00

(52) U.S. Cl. CPC G06F 19/3418; A61B 5/4821; A61B 5/021; A61B 5/6898; A61B 5/743; A61B 5/7475; A61B 5/7246

(57) ABSTRACT

An Operating Room Management System (ORMS) for facilitating the efficient and optimal organization of hospital medical personnel and resources runs on local and remote servers and comprises a real-time updated daily schedule which is displayed on digital whiteboards and other displays throughout the hospital. The schedule display simplifies the crucial information concerning common hospital concerns and permits quick, accurate decision making on the part of the hospital medical personnel and administrators. A digital version of an anesthesia monitoring chart on a tablet or other mobile device is used to record patient vitals and other important data during operations.

### FIELD OF THE INVENTION

This application relates to healthcare facility management software and more particularly to operating room management system (ORMS) software having an interacting mobile application for anesthesia monitoring.

### BACKGROUND

Hospitals and other healthcare facilities providing surgical services must coordinate a myriad of resources, medical personnel, and hospital staff to provide optimum and efficient care to their patients. Information about status of these resources and the facilities' patients must be updated constantly and be available to the relevant medical personnel and facility staff in the operating rooms (ORs) where the surgical services are delivered, in other ancillary rooms of the facility, and to medical personnel and facility staff who may be in remote locations. Particular medical personnel or facility staff members may need to be alerted with respect to the updated status of particular resources or patients and may need to provide updates to the information displayed from where ever they are located.

Modern hospitals are complex, technologically sophisticated organizations having sometimes thousands of employees, doctors, nurses, medical technicians and administrators, with critical life or death decisions being made regularly—and sometimes having to be made abruptly and quickly. Up-to-date, easily apprehended information about the personnel and resources available can make a difference. And even when critical decisions are not at stake, the recent increases in the cost of health care have made it imperative to use the facility personnel and resources as efficiently as possible.

U.S. Pat. No. 5,842,173 to Strum et al., issued for “Computer-Based Surgical Services Management System,” describes a complex database running on a server and display system to coordinate surgical services at a medical center, but it is updated only periodically, not continuously. It also does not provide for remote notification and interaction by the medical center personnel.

U.S. Pat. Applications No. 2010/0306858, 2010/0305970, 2010/0305971, 2010/0305972, and 2010/0305973 to McLaren et al. describe central medical information systems with interacting mobile applications. The described systems, however, are based on a typical medical case or patient focused database and display, rather than systems focused on the status and coordination of medical facility resources.

U.S. Pat. No. 7,657,445 to Goux describes a system for managing healthcare facility resources, but it is specifically focused only on tracking the number of hours provided per patient rather than on the coordination of the large number of resources used by a typical healthcare facility or giving medical personal the information they need to make quick and accurate decisions.

### SUMMARY

In accordance with the present disclosure, embodiments of a system, method, and apparatus are described which eliminate or ameliorate the problems and disadvantages associated with previous systems, methods, and apparatuses.

According to a particular embodiment, a system of servers is provided, which communicate data in real time with white boards stationed in the operating rooms of a healthcare facility. Medical personnel and healthcare facility staff members can view the formatted data on a white board and input new or revised data directly on the white board or at an input station near the white board.

Further in this embodiment, mobile phones carried by medical personnel and healthcare facility staff run applications enabling the real time display of data communicated by the servers and allowing the input of new or revised data into these mobile phones to be transmitted to the servers and displayed when appropriate on whiteboards and other displays throughout the facility. The mobile phone applications further permit the alerting of specific personnel, other mobile phone carriers and throughout the facility.

### BRIEF DESCRIPTION OF THE DRAWINGS

FIG. 1 is an illustration of a hospital daily schedule display on an operating room digital white board.

FIG. 2 is an illustration of the team building display on an operating room digital whiteboard in an embodiment of the invention.

FIG. 3 is a block diagram detailing the arrangement of specific servers in an embodiment.

FIG. 4 is a block diagram illustrating the arrangement of specific servers within the cloud server in another embodiment of the invention.

FIG. 5 is a block diagram illustration of patient movement through the hospital and corresponding data entry.

FIG. 6A is an illustration of tabs on the daily schedule display indicating the availability of viewable side panels.

FIG. 6B is an illustration of a displayed side panel on the daily schedule display.

FIG. 6C is an illustration of a second displayed side panel on the daily schedule display.

FIG. 6D is an illustration of a third displayed side panel on the daily schedule display.

FIG. 7A is an illustration of a stop light alert icon in an embodiment.

FIG. 7B is an illustration of a stop light alert icon in a small window on the desktop display of an administrator.

FIG. 8 is an illustration the main navigation display on the smartphone running the ORMS app of an embodiment.

FIG. 9 is an illustration of the staff contact display on the smartphone running the ORMS app of an embodiment.

FIG. 10 is an illustration of a typical anesthesia record form for real-time monitoring of anesthesia administration during an operation.

FIG. 11 is an enlargement of the monitoring graph portion of the anesthesia record form of FIG. 10.

FIG. 12 shows an enlargement of the symbol legend of the monitoring graph portion of FIG. 11 so that the descriptions for the various symbols used on the monitoring graph may be read.

FIG. 13 is an illustration picture of a typical monitoring graph after use in an operation.

FIG. 14 is a depiction of an anesthesia monitoring graph displayed on a mobile device screen in an embodiment of the invention.

FIG. 15 is a depiction of a used anesthesia monitoring graph displayed on a mobile device in an embodiment of the invention.

### DETAILED DESCRIPTION

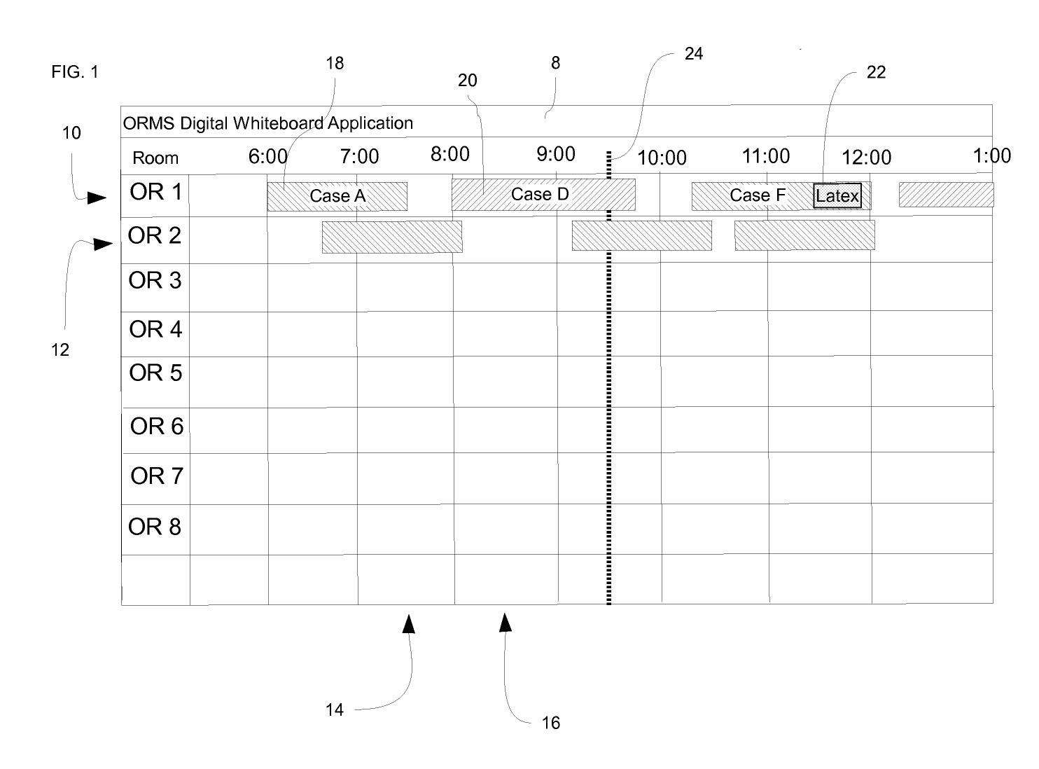

In an embodiment of the invention, FIG. 1 is an example illustration of a daily schedule 8 for the operating rooms of a hospital or healthcare facility. Schedule 8 would be displayed on a multiplicity of digital whiteboards in various places of the healthcare facility such as the anterooms of the operating rooms. The rows of schedule 8 correspond to different operating rooms of the facility. For example, rows 10 and 12 correspond to OR 1 and OR 2 respectively as indicated. The columns, for example 14 and 16, correspond to the time of use of the operating rooms by the patient cases undertaken. The current time of day is indicated by a vertical line 24 through schedule 8.

The patient cases scheduled are depicted as shaded or colored blocks, for example 18 and 20, extending for the length of time they will occupy the operating room. Text within the blocks, such as shown 18 and 20, provide details of the cases such as the name of the patient and the procedure planned. The type of shading or different colors of the blocks 18 and 20 indicate the case status and current location of the patient. Alerts concerning the case such as pertinent allergies may also be indicated by a separate shaded or colored area with a case block as illustrated at 22 for example. Such a color-coded, resource-focused schedule in combination with a display of simplified information is unique in the setting of a hospital or health-care facility and is surprisingly effective in providing a comprehensive picture of the data needed to optimally organize the doctors, nurses and other health care personnel, and the resources they need, and further, uniquely and surprisingly enhances the making of quick, accurate decisions in critical situations.

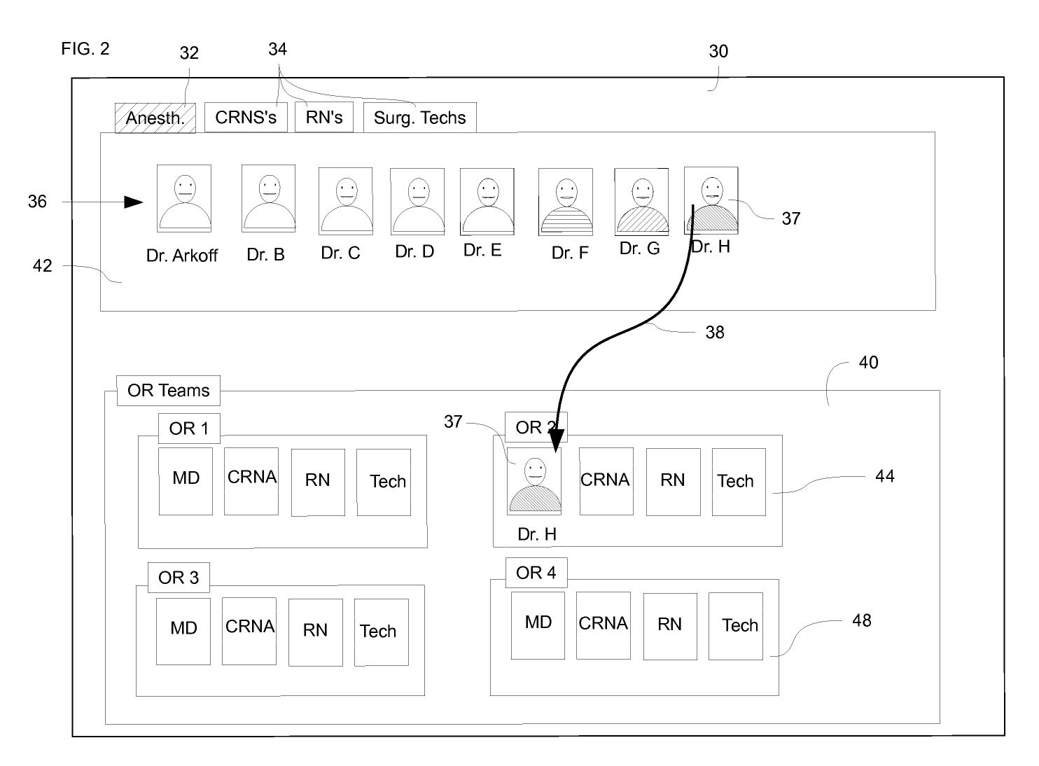

Surgical teams can be formed and assigned easily using a display 30 of individual health care facility personnel organized by specialty and work shifts such as illustrated in FIG. 2. In this embodiment, medical personnel by specialty are shown in a designated window 42 at the display top. In this embodiment, the group of anesthesiologists have been selected to be displayed by highlighting tab 32. Once selected, the anesthesiologist's names and pictures are displayed in a row as indicated 36. Alternatively, if any of tabs 34 were selected and thus highlighted, the corresponding group of medical personnel would be displayed in the window 42.

To form a particular team, individual personnel available to work that day are dragged and dropped to the appropriate position in the chosen team in lower box 40. As an example, in FIG. 2, the picture of Dr. H (numbered 37) has been selected using a computer mouse, touchscreen, or other computer input device and has been dragged and dropped on the MD position on the OR 2 display area (numbered 44) as figuratively indicated by arrow 38. Depending on their availability, personnel can be assigned to more than one teams. In such a case, the picture and name of the person remains in the top row until he has been assigned the maximum number of times. This is shown by example in this embodiment with the photo of Dr. H (numbered 37) remaining in the top row 36 at the same time his photo is displayed as a part of OR 2 (numbered 44). The maximum number of times a person can be assigned to teams is typically determined on an individual basis.

Once formed, the teams are dragged and dropped on particular patient cases and then displayed on the main schedule 8 on whiteboards and other displays throughout the facility. The dragging and dropping process can be carried out directly on the whiteboard or other display using a method of digital input such as computer mouse or stylus, or to facilitate quick and easy input to the display on a digital whiteboard, a user can use for example a tablet computer communicating with the whiteboard via a bluetooth or other wireless connection and displaying a facsimile of the team building screen.

A suitable digital whiteboard capable of displaying the schedule 8 is the Hitachi Starboard FXTRIO Interactive Whiteboard available from Hitachi Solutions America, Ltd., 601 Gateway Blvd. Suite 100, South San Francisco, Calif., although many other digital whiteboards or other computer-controlled display systems can be used. If the Starboard FXTRIO digital whiteboard is used, then a software interface such StarBoard Software also available from Hitachi Solutions can be used to facilitate the creation and modifications of schedule 8.

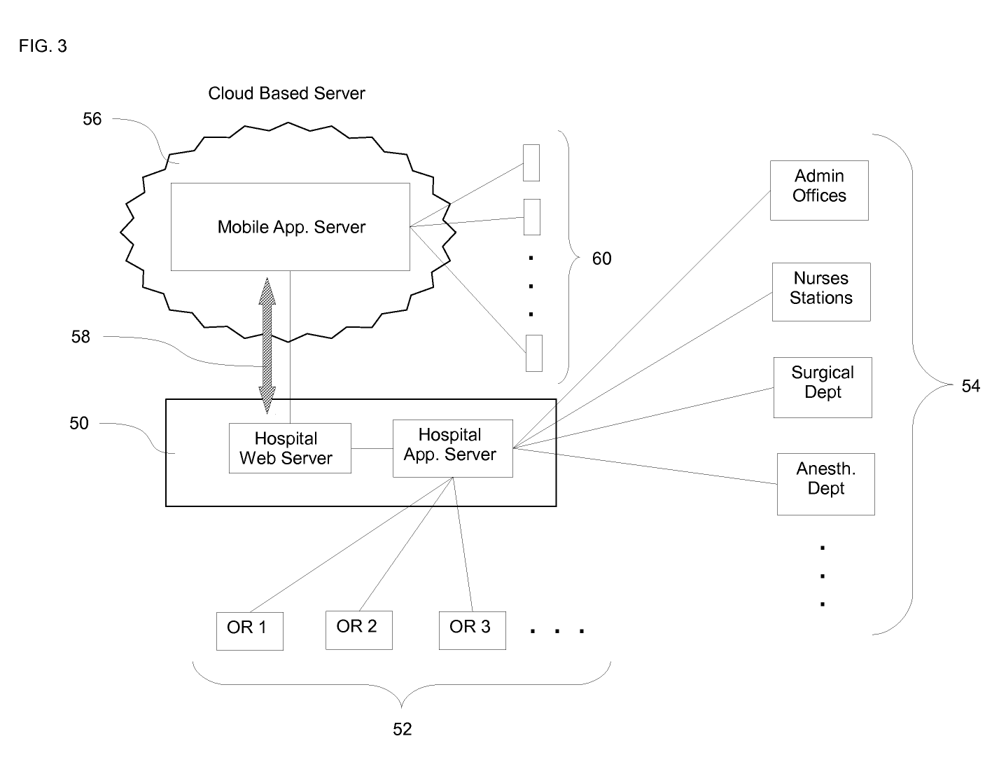

FIG. 3 is a block diagram overview of the overall system running the ORMS software in an embodiment. Local server 50 receives, stores, calculates, and transmits data in forms such as schedule 8 to the facility operating room sites 52 and facility offices and stations 54. Schedule 8 and other data may be displayed on for example digital whiteboards, desktop displays, tablet computers, or mobile devices depending on the needs of the site or office. The local server 50 is in continual, real time communication with cloud-based server 56 which is not physically located at the healthcare facility. In an alternative embodiment, all variable patient, case and resource data is maintained on the local and cloud-based servers and downloaded to the individual whiteboards, displays, stations, or departments, but calculation and formatting of the schedule and determination of alerts is done by applications or software modules running on the PC's or embedded computers associated with each individual whiteboard, display, station, or department.

All substantive data on local server 50 is continually backed up on cloud-based server 56 and vice versa as indicated by arrow 58. In this embodiment, the data continually backed up includes all current information about the patient cases the hospital has undertaken and the information about the health care facility resources and personnel necessary to calculate and display the schedule.

Cloud-based server 56 transmits a facsimile of the schedule 8 and other data to apps on the mobile phones 60 of doctors and other relevant health care personnel. Also, via this path, alerts can be texted or otherwise transmitted to specific personnel. An alert is short message or datum of high importance and urgency. Alerts may for example indicate an unexpected problem or delay with a particular patient or case, or patient overcrowding at a particular stage or location within the facility. Alerts can be manually triggered for example by personnel at any of the offices and stations 54, or automatically triggered by one of the servers based on calculations from data input by personnel at for example operating room sites 52 or offices and stations 54. Such automatically triggered alerts can be fixed as a part of the system design or can be customized by various healthcare facility personnel.

An alert displayed on one or more mobiles phone 60 can be responded to immediately by a user or users and data in the response displayed in real time on one or more of the digital white boards near the OR's 52, station or office desktop displays 54 or other displays in the healthcare facility or in the overall system. The response can be a direct change in the displayed schedule or used to automatically calculate a change in the schedule which is then displayed. Personnel at these sites can then make further adjustments to the schedule or input other data accordingly. Correspondingly, any changes in the schedule such as illustrated FIG. 1 will be transmitted in real time to the other sites and the apps on the mobile phones 60. Having the capability of alerts for mobile users which can be responded to by transmitting schedule changes to the overall system is a unique and surprisingly effective method of optimizing the resource usage of the hospital or health-care facility.

Local server 50 can be implemented using a standard PC with for example an Intel Haswell microprocessor running the Windows 8 operating system. Of course Apple or UNIX-based computers, among others, could also be used as would be obvious to engineers with ordinary skill in the art. Cloud-based server 56 can be implemented for example using a commercial cloud computing service such as Amazon Web Services available at URL http://aws.amazon.com/ or using standard PCs at a remote location. Software development for the servers and the station or desktop modules can be done in Visual Basic with the Microsoft Visual Studio development environment although myriad other programming languages and development environments can be used. The displays such as illustrated in FIGS. 1 and 2 are typically implemented using an internet browser such as Firefox and coded in HTML although other browsers or direct implementation in Visual Basic or many other programming languages and development environments known to software engineers with ordinary skill in the art can be used.

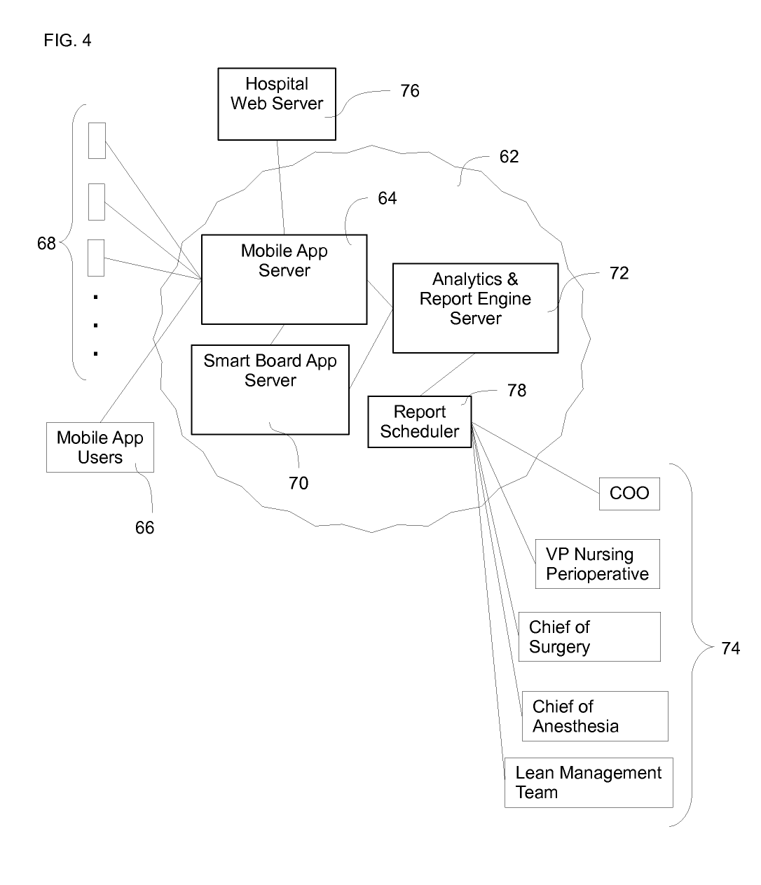

FIG. 4 is a block diagram of an arrangement of application-specific servers at remote cloud server 62 in another embodiment of the invention. In this embodiment, cloud server 62 includes a mobile applications server 64 which communicates directly with the mobile phones of users running an app 66. The mobile applications server can also send and receive SMS text messages to the mobile phones 68 of users whose phones do not have the mobile app capability.

Cloud server 62 also runs the digital white board application server 70. Both the digital white board server 70 and the mobile applications server 64 communicate data directly to the analytics and report engine server 72 which analyzes said data and creates appropriate reports. Said reports are sent periodically via the report scheduler 78 to the appropriate personnel, collectively 74, at the hospital. Generally, realtime data is communicated to and from the hospital display locations, stations, departments, and offices via the hospital web server 76.

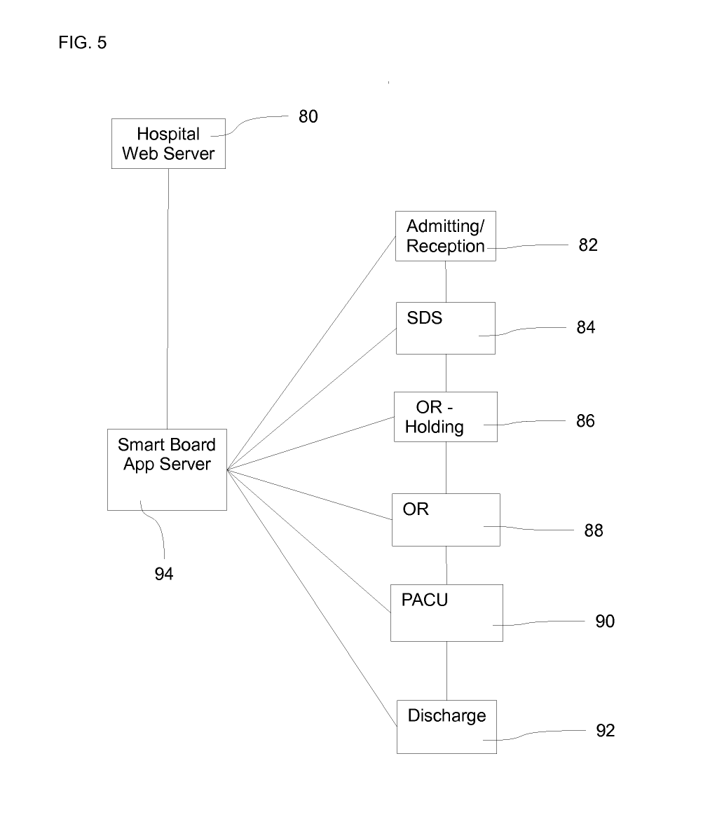

FIG. 5 is a block diagram showing patient movement through the health-care facility. In this embodiment, when a patient enters the reception area 82, his arrival is input to the hospital web server 80 by admitting personnel and his case is displayed on the OR schedule such as illustrated 18 and 20 for example on FIG. 1. As the time for the patient's operation approaches, he moves to SDS 84 and then to the OR holding area 86.

At each department or station, the patient's progress is updated on the OR schedule by the staffing personnel who typically input the data using desktop PCs or tablet computers. The ORMS software module running on the desktops PCs or tablet computers is typically customized for each department or station.

At the appropriate time and when the surgical team and all resources are ready, the patient moves to the OR 88 and the operation is performed by the surgical team. After the operation is complete, the patient moves to the PACU 90 and when ready the discharge area 92 where the patient may fill out a survey which he inputs directly to the hospital web server 80.

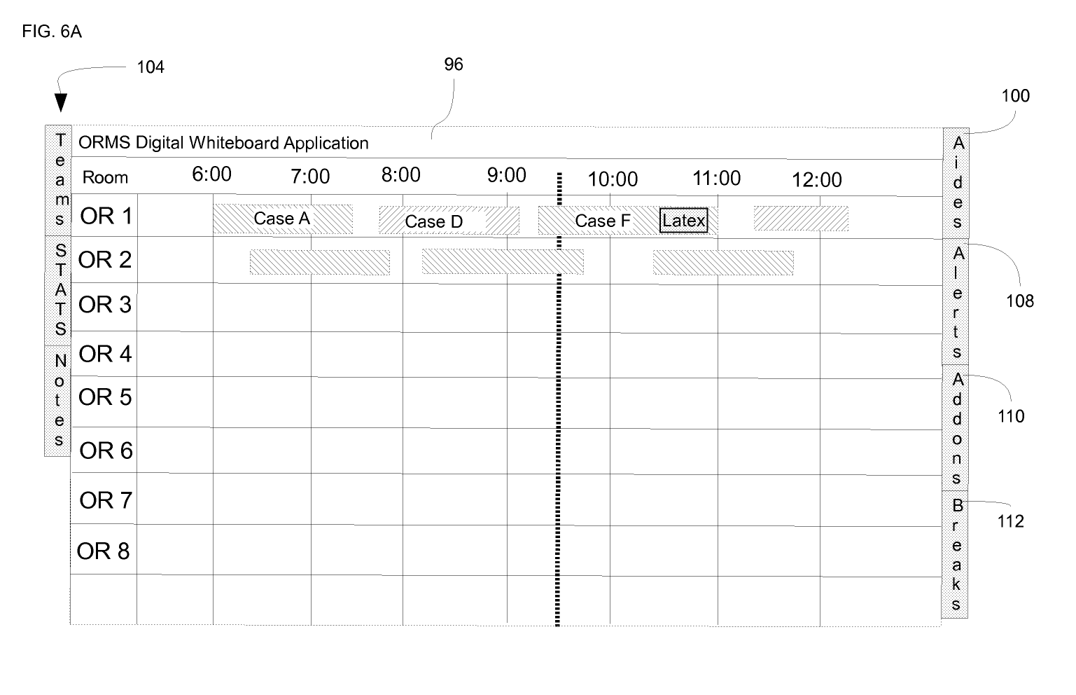

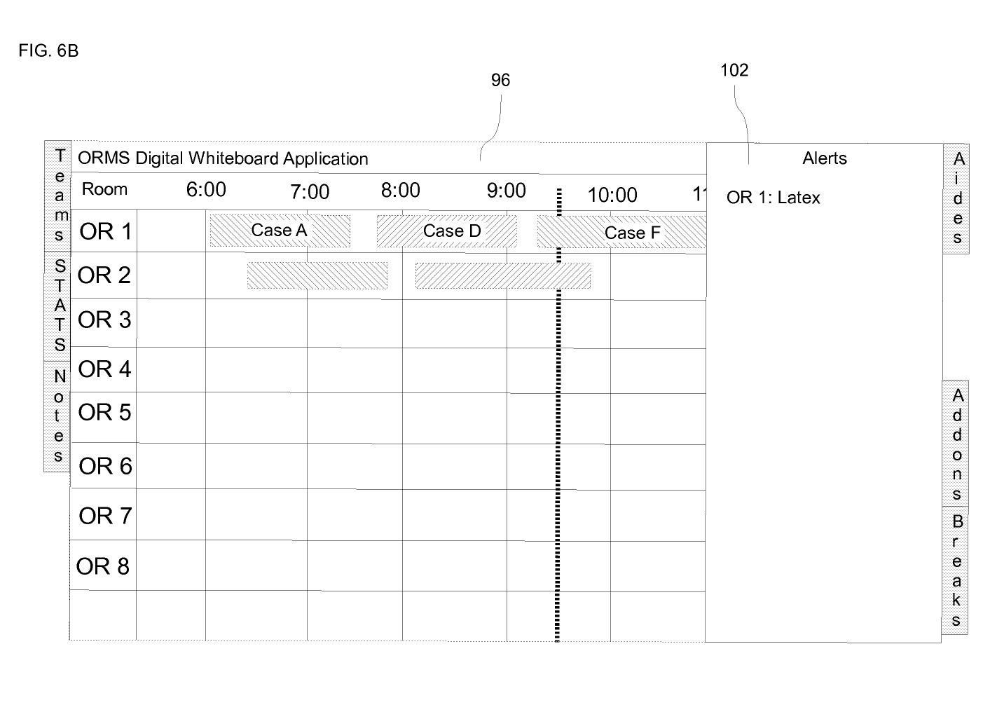

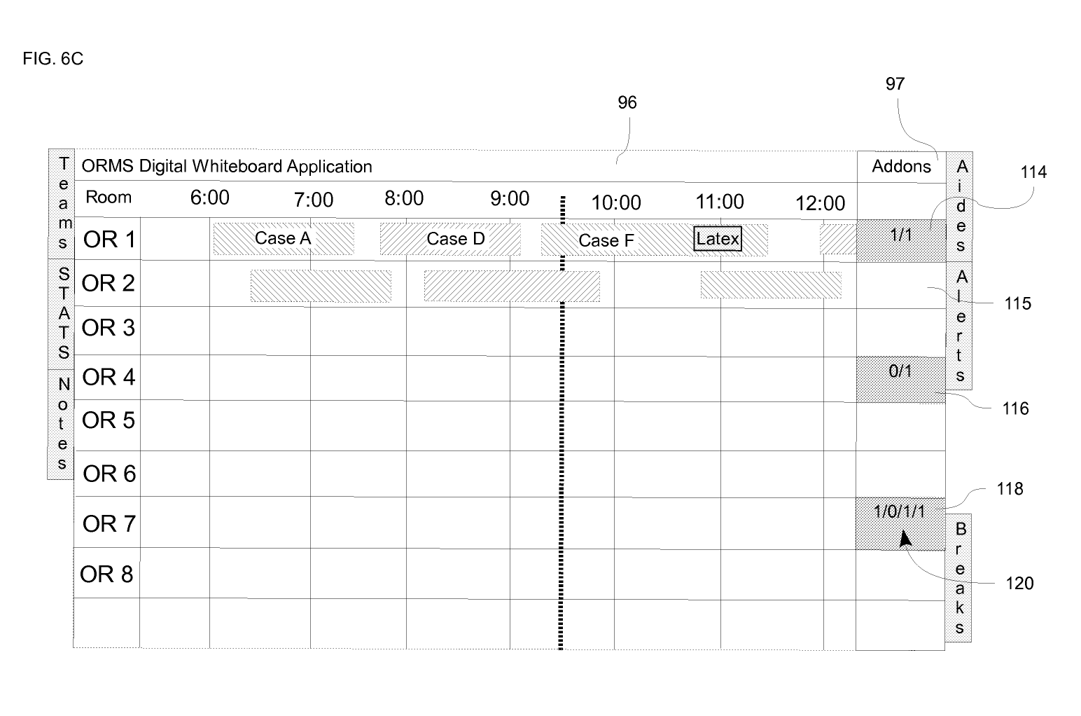

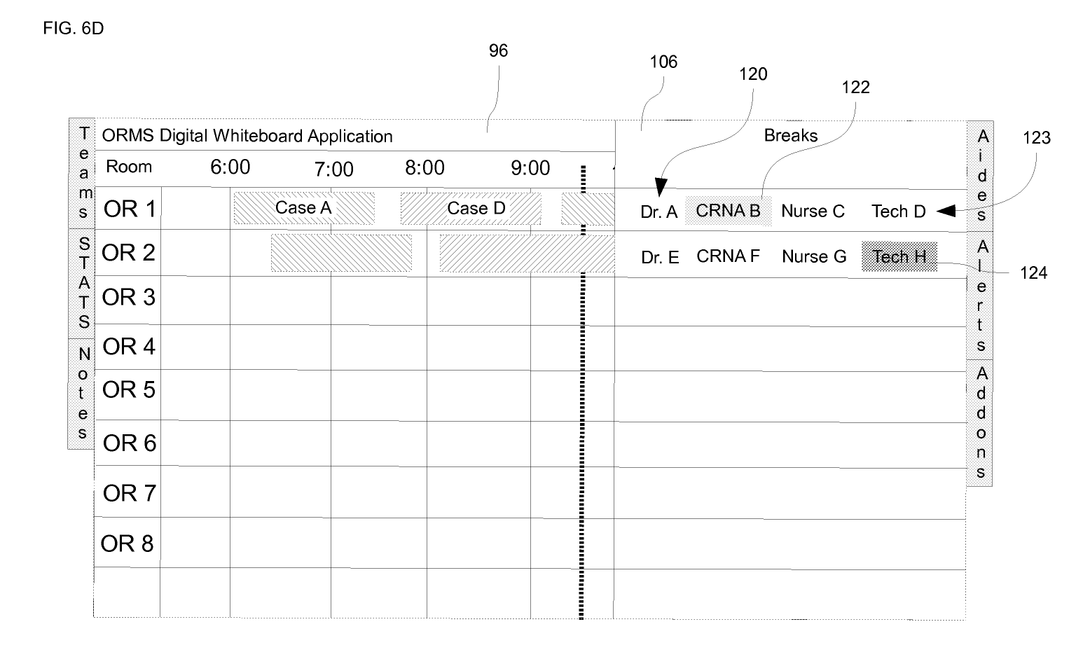

In another embodiment, FIGS. 6A to 6D illustrate dockable slide-out panels on the OR schedule. In FIG. 6A, Tabs 100, 108, 110, and 112 arrayed on the right hand side of the daily schedule display 96 have labels indicating available panels with pertinent information. Other such tabs, as indicated at 104, are arrayed on left hand side of schedule 96. In FIG. 6B, the Alerts tab 108 has been selected using for example, a computer mouse or touch screen entry, and the alert panel is displayed 102 while Alerts tab 108 temporarily disappears.

Information or notes can be added directly to panel 102 with digital input, for example, keyboard or computer mouse, and this information or notes is reproduced on some or all schedule displays through the hospital depending on user-selectable parameters. In an alternative embodiment, information or notes can be written directly in panel 102 area in an analog manner, that is, with a stylus or even a finger, and this information or notes is reproduced on some or all schedule displays throughout depending upon selectable parameters.

FIG. 6C is an illustration of the schedule 96 when the Addons tab 110 has been selected such that the Addons panel 97 is displayed. An addon is a case which is newly added after the schedule for the day is created, with corresponding allocation of the hospital personnel and resources, and which is expected to run past a hospital's employee shift change time. Another type of addon is a case which was on the daily schedule when created but is delayed or running late due to unforeseen circumstances and is thus expected to run past the time of one or more employee shift changes. It is important for hospital managers and administrators to know quickly and accurately how many addon cases they have and through what shift changes they will go so they can make sure they will have the personnel present and resources on hand to cover these cases. A hospital or other health-care facility may have several shift change times when nurses, technicians, and other medical and administrative personnel end or start their work day. For example, a hospital might generally have four shift change times at 3:00 PM, 3:30 PM, 5:00 PM, and 7:00 PM.

For example, in FIG. 6C, block 114 is highlighted to indicate there is one or more addon cases in OR 1. Block 116 is highlighted to indicate there is one or more addon cases in OR 4. And block 118 is highlighted to indicate there is one or more cases in OR 7. When no addon cases have been added to the schedule for an operating room, the corresponding addon block is not highlighted, for example, 115, and remains the neutral color of the schedule, typically white.

The highlighted addon blocks, 114, 116, and 118, contain a simple symbolic code, such as seen at 120 for example, which indicates how many addon cases are scheduled for the corresponding OR and the shift change times they are expected to run past. In this embodiment, the symbol 1 is used in the code to indicate an addon case that will run past a shift change. The position of the symbol 1 in the code indicates which shift change the addon case will run past. So in the case of a hospital with four employee shift changes at 3:00 PM, 3:30 PM, 5:00 PM, and 7:00 PM such as described hereinabove, the code would have up to four positions, reading from left to right. So for example, the code 120 has a 1 in first position indicating a case which will run past the earliest shift change time of concern, 3:00 PM. Then there is the symbol ‘/’ as a separator and then the symbol 0 as a placeholder, indicating that there are no addon cases in OR 7 anticipated to run past the 3:30 PM shift change. Continuing to read from left to right at 120, there is a second ‘/’ and then a 1 indicating that there is an addon case expected to run past the 5:00 PM shift change time. Finally, there is a ‘/’ and a 1 indicating that there is an addon case expected to run past the 7:00 PM shift change. Note, it may or may not be that the addon case running past the 7:00 PM shift change is the same case or a continuation of the same case that is expected to run past the 5:00 PM shift change as described hereinabove.

Thus, the combination of color highlighting 114, 116, and 118, and a simple symbolic code 120, enables a hospital administrator or medical personnel manager to at a glance quickly and accurately determine the additional personnel and resources needed to timely complete, with optimum outcome, the cases the hospital has undertaken. That this combination of color highlighting and an associated simple symbolic code on a comprehensive, real-time updated daily schedule displayed or available to display at a multiplicity of locations throughout the hospital or healthcare facility, provides such facile comprehension of a possibly critical situation at hand, permitting quick and accurate decision making by perhaps a multiplicity of hospital or health-care facility administrators and managers at different locations, is a unique and surprising, perhaps even revolutionary, result.

FIG. 6D is an illustration of the schedule 96 when the Breaks tab 112 has been selected such that the Breaks panel 106 is displayed. A break is a relatively short period during the work shift of a hospital or healthcare facility employee when they are “off-duty”, perhaps having a meal or a rest-break. The current break status of an employee is displayed and can be modified in the Breaks panel 106.

The personnel on a particular OR team are displayed in the row corresponding to the OR to which they are assigned such as indicated at 123. The name of team member not on break and working normally, indicated at 120 for example, will have a background of a neutral color, typically the same as the neutral color of daily schedule, typically white. A team member name can be selected, such as by clicking with a computer mouse or tapping with a finger or stylus on a touch-sensitive display, and then the background of the team member name will turn a color, for example 122, understood to indicate they are on break. A team member name can be selected a second time, and the background of their name will turn a second color, for example 124, understood to mean they have returned from break and are now working normally. After a set period, perhaps a few minutes, which can be configured by hospital administrators or other personnel, the second colored background 124 will automatically change back to the original neutral color indicating normal working status 120.

The organizational efficiency and decision making confidence gained by having all this information about the hospital's current patient cases available in a single view display of the daily schedule of the hospital is unique and surprising. However, while the hospital administrators, like the hospital medical personnel, benefit from having available comprehensive information about the hospitals current cases and relevant resources, not every administrator needs to have a continuous view of the schedule display.

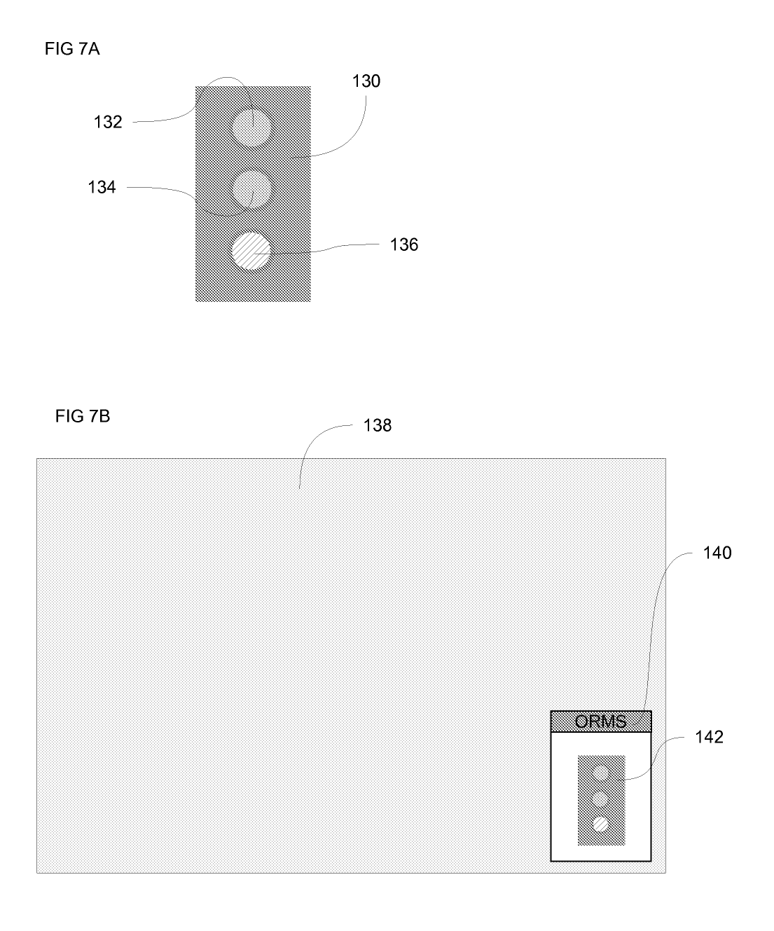

FIGS. 7A and 7B are illustrations of a stoplight icon 130 which can be displayed on the computer desktop display 138 of administrators and at stations where the OR schedule display is not continuously needed. In a fashion similar to the workings of an ordinary traffic light for the control of street traffic, when there are no known problems likely to interfere with hospital's daily schedule as planned, the upper “lights” 132 and 134 would be dimmed and lower “light” 136 would be highlighted, typically green. If an alert condition occurs, which can be manually triggered or automatically triggered by calculation as described hereinabove, then light 136 would be dimmed and upper lights 134 or 132 typically would be yellow or red respectively, depending on the urgency of the alert.

As shown in FIG. 7B, stop light icon 142 would typically be shown in a small window 140 on an administrator's PC desktop 138 but in other embodiments the icon could stand alone on the desktop without a surrounding window or in many other configurations as would be apparent to software engineers of ordinary skill in the art.

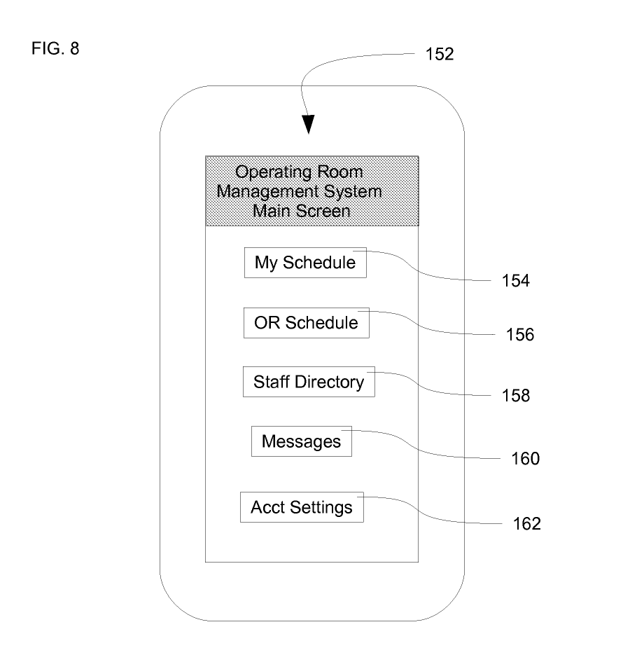

FIG. 8 is an illustration of the main navigation screen of the ORMS app on the smartphone of a user who might typically be a doctor or nurse at the hospital. The smartphone display indicated 152 shows selectable options to display the user's personal schedule 154, the OR daily schedule 156, the staff directory 158, the user's messages 160, and the user's account settings 162.

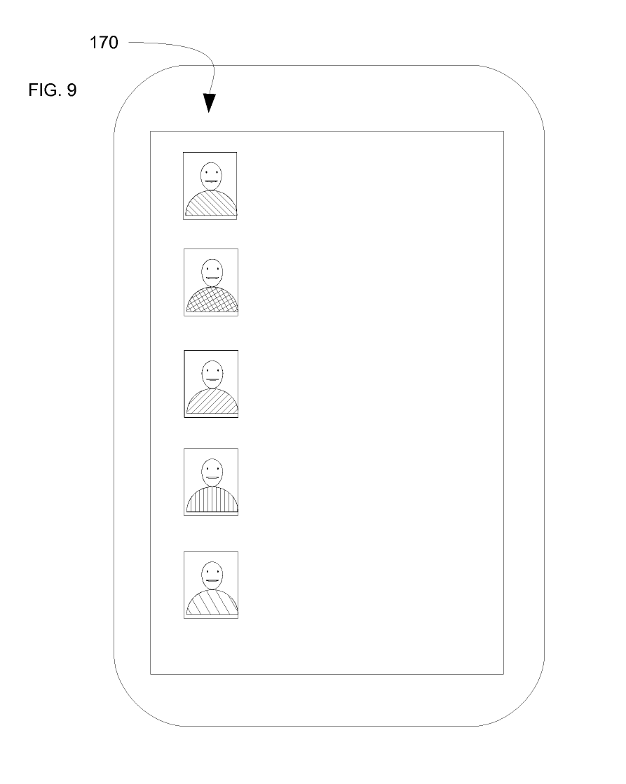

FIG. 9 is an illustration of the staff directory display in an embodiment of the ORMS app on the smartphone or other mobile device of a user who might typically be a doctor or nurse at the hospital. The smartphone or mobile device displays a column 170 of pictures of medical facility staff members or other important contacts. Simply touching, clicking or otherwise actuating the picture of the desired contact allows the user to generate a text message, phone, or other otherwise contact or alert the pictured staff member.

If the smartphone or other mobile device of the user can be used to take a picture of the user, that picture can be directly uploaded to the ORMS network and automatically propagated to the displays of all other ORMS users for use on their staff directory displays.

In another embodiment of the invention, a “smartchart” tablet or mobile device application or app is used to record patient vital signs monitored during an operation in order to facilitate calculation of trends and other analysis of data over the course of the operation and to transmit the data in real-time to the supervising anesthesiologist and the hospital database associated with server 50 which includes the Electronic Medical Record (EMR) of the patient.

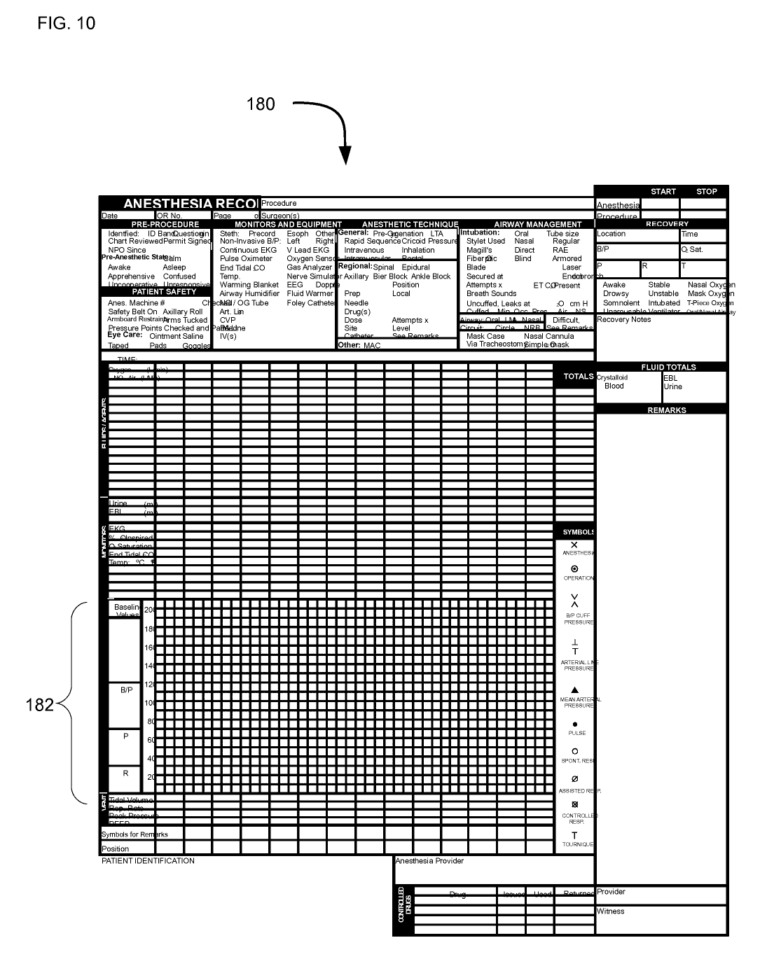

FIG. 10 is an illustration of a typical anesthesia monitoring record form, generally indicated 180. During an operation, the anesthesiologist, nurse anesthetist, or other medical personnel administering the patient anesthesia, referred to as the anesthesia administrator, typically will record the anesthesia data and parameters, patient data and vital signs, and other relevant medical events occurring during the operation on a paper version of such a record form 180. The form 180 or a facsimile of it becomes a part of the hospital's (and patient's) medical records.

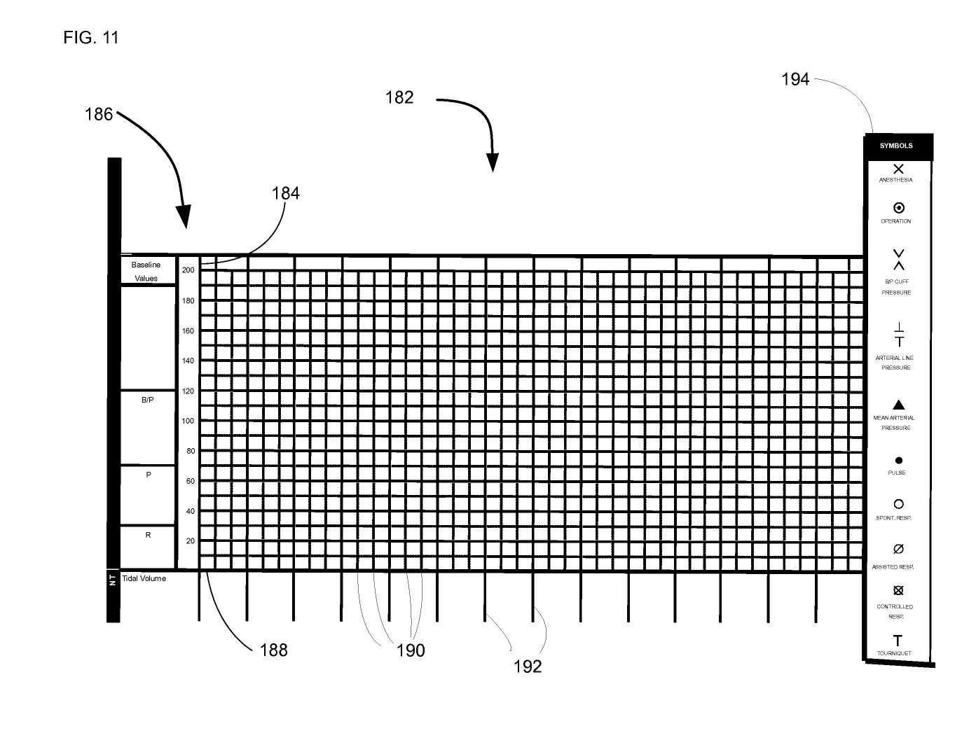

The vitals charting or graph section of the anesthesia monitoring record form is indicated 182. FIG. 11 is a view of the vitals graph section 182 enlarged to show detail. The graph ordinate axis 184 labeled in intervals of 20, as indicated 186, is used to indicate the magnitude of the charted patient's vital signs and measurements. The graph abscissa axis 188 is typically not labeled but is typically understood to be scaled such that the minor divisions, a few indicated 190 for example, signify 5 minute intervals during the operation, and the major divisions, two of which are indicated 192, signify 15 minute intervals during the operation.

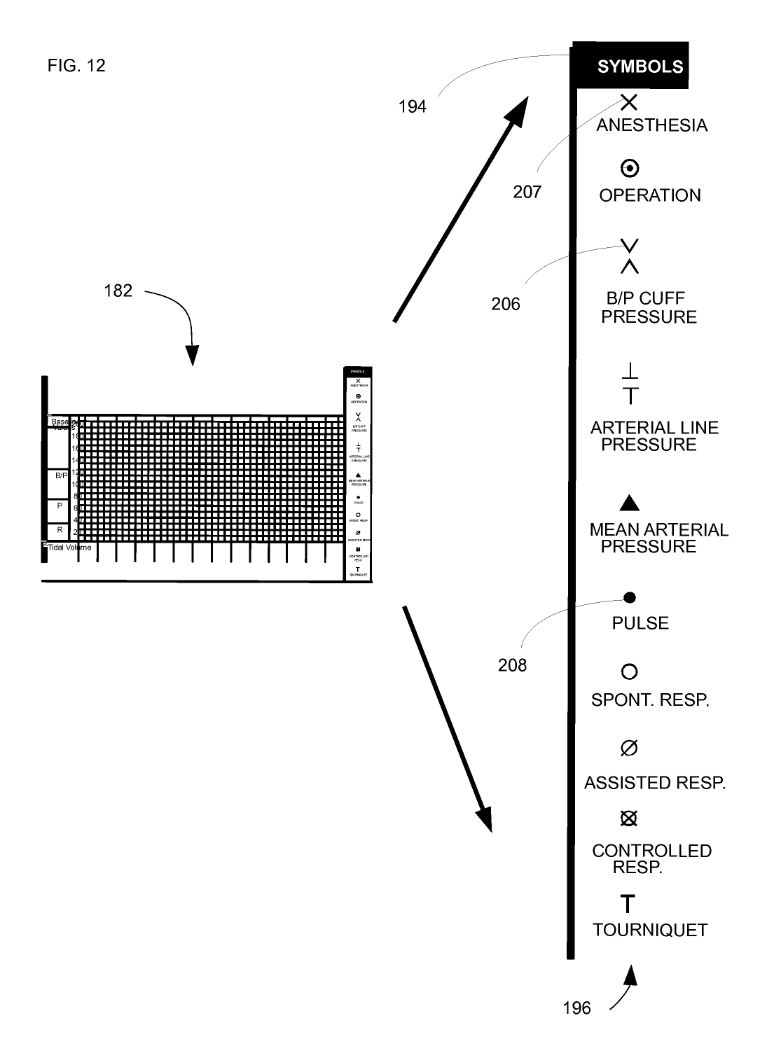

On the right hand side of graph 182 is the symbol legend 194. FIG. 12 shows an enlarged view of symbol legend 194. The symbols such as indicated 196 are used widely and worldwide for anesthesia monitoring but other symbols may be used. Typically the anesthetist, nurse anesthetist, or other anesthesia administrator will record by hand the measured patient vital signs on a paper version of such an anesthesia graph 182, plotting the corresponding symbols such as indicated 196 on graph 182 with the position or coordinates of the symbols on graph 182 indicating the time and value of the vital sign measurement by comparison to the graph axes, the abscissa 188 and the ordinate 184.

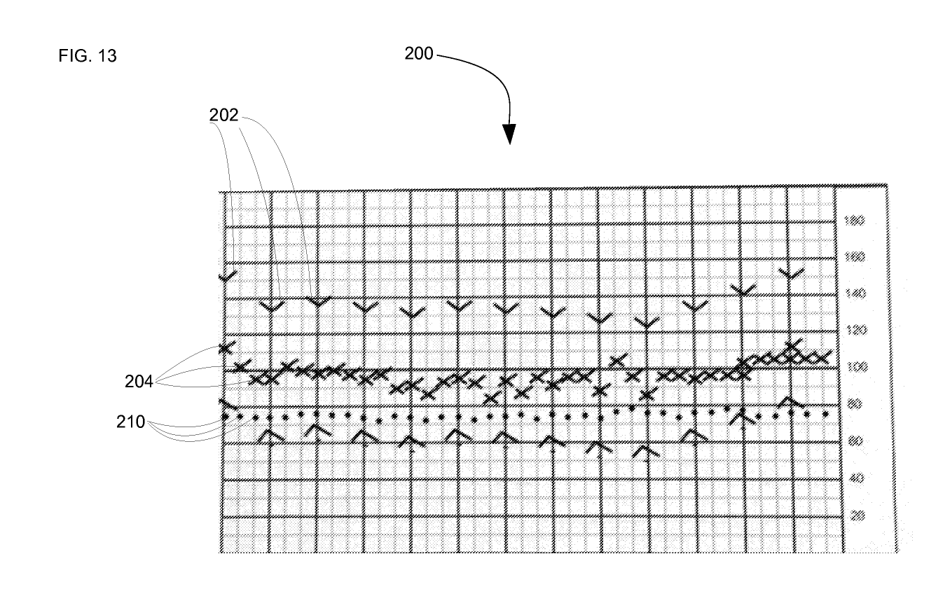

FIG. 13 is an example of a typical anesthesia graph 200 which has been used to record patient vital signs during an operation. Blood pressure readings such as indicated for example 202 were recorded every 15 minutes during the operation using the designated symbol 206 from symbol legend 194. The anesthesia level was checked or adjusted every 5 minutes with the reading recorded as indicated for example 204 using the designated symbol 207, and the patient's pulse reading was checked every 5 minutes and recorded as indicated for example 210 using the designated symbol 208 from the symbol legend 194.

After the operation, graph 200 typically becomes a part of the hospital or medical facilities permanent records. It may also be scanned in or digitized in known ways to become a part of the hospital's electronic medical records database.

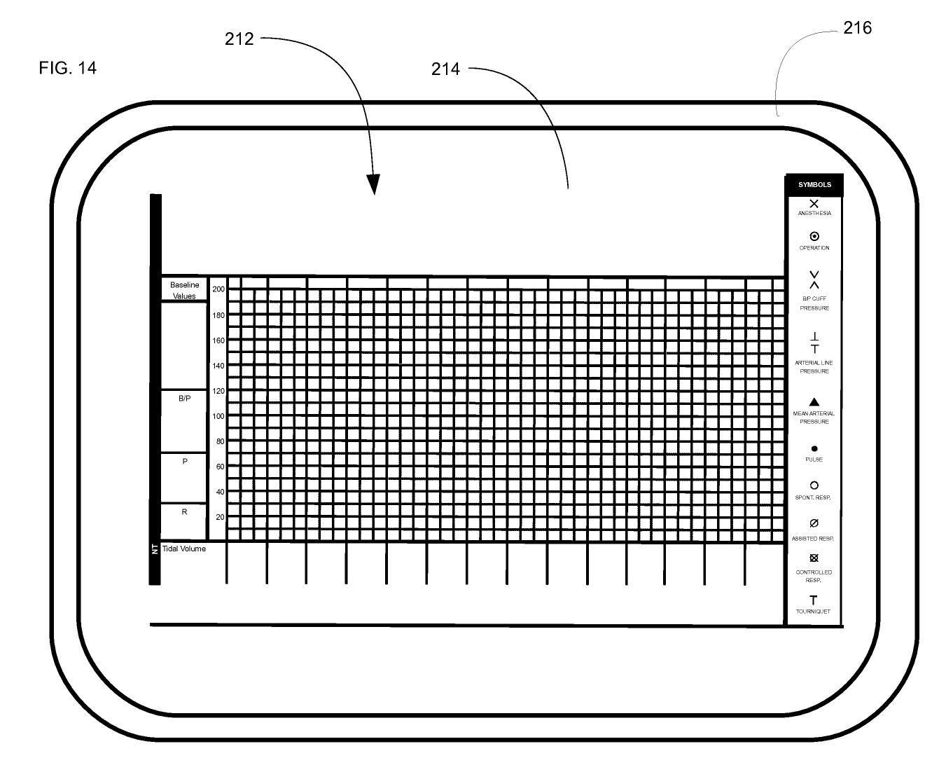

In an embodiment of the invention, FIG. 14 is a depiction of an anesthesia monitoring graph, such as 182, here indicated 212, but displayed on a mobile device 216 such as a tablet computer, for example, an Apple iPad or Google Nexus, with a touch sensitive screen 214. The anesthesia administrator can draw symbols, such as shown in symbol legend 194, or other notes or notations on the mobile device screen using a stylus such as known in the art. In a preferred embodiment of the invention, during an operation on a patient, the anesthesia administrator records the patient's vital sign measurements and other important data directly on the touch sensitive screen 214 with a stylus using standard symbols such as shown in the symbol legend 194.

In this embodiment, the symbols or other marks displayed on touch sensitive screen 214 are rendered as exact replicas of the marks drawn on the screen by the administrator. The mobile device app does not in any way interpret or modify the marks, instead simply recording and displaying them faithfully as rendered by the anesthesia administrator for the duration of the patient's operation.

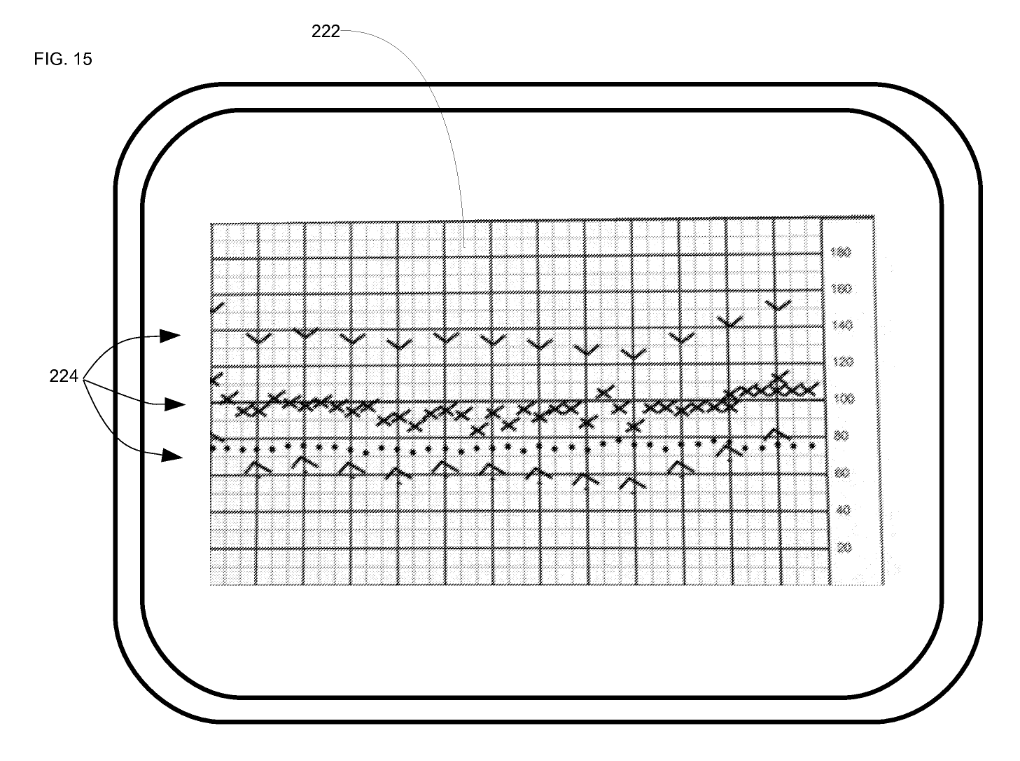

However, in this embodiment, the mobile device or tablet computer 216 is in wireless communication with hospital server 50 and transmits the marks such as 224 on FIG. 15 and their positions or coordinates relative to the graph axes 188 and 184 to the server 50 which uses a software handwriting recognition SDK such as available from Microsoft Corporation to interpret the marks made by the anesthesia administrator as vital sign symbols and, in conjunction with the symbol positions or coordinate information, determines the vital sign values and can act automatically, for example sending an alert to hospital personnel if the patient's blood pressure vital sign values exceed or fall below predetermined thresholds. A final version of the anesthesia record is also automatically produced for inclusion in the patient and hospital EMR after review by the anesthesia administrator.

Another embodiment can be envisioned where the marks made are interpreted as symbols or perhaps letters by the app or the hospital network with which the app communicates, and these symbols or letters are then displayed during the operation on a chart such as 212 in the interpreted position apparently intended by the anesthesia administrator. But if the symbol or symbol value interpretation is inaccurate, then other medical personnel could make errors as a result. Thus as described hereinabove, in a preferred embodiment, the marks made by the anesthesia administrator are recorded without modification and faithfully reproduced on tablet 216 for the duration of the patient's operation but simultaneously communicated to the hospital or medical facility server 50 for essentially instantaneous (or “real-time”) and continuous detailed computer interpretation.

Further, in the preferred embodiment under discussion, while marks made by the anesthesia administrator are not interpreted for the purpose of correcting their display or communicating corrected marks to the hospital or medical facility database, the marks made may in fact be interpreted by other network software on server 50 for the purpose of trend analysis or calculation. For example, the anesthesia administrator typically records blood pressure readings using symbol 206 or similar every five minutes during an operation but if they are unduly busy or otherwise distracted they may not notice long term trends such a slow drop in blood pressure which in some cases could be injurious to the patient. In this preferred embodiment, the server 50 in communication with mobile device or tablet 216 may interpret certain marks made on chart 212 to be symbols associated with blood pressure readings such as 206 and automatically calculate the trend in the readings over the course of the operation. If, according to predetermined thresholds, the trend might typically be of concern to the anesthesia administrator or other medical personnel, then a text, symbolic, or audible alert can be sent so that the significance of the trend analysis can be competently evaluated.

The marks such as made on graph 212 by the anesthesia administrator can be automatically communicated instantaneously or in real-time to a supervisor or other medical personnel in another location for review. The reviewing supervisor or other medical personnel can then communicate questions and concerns with respect to the ongoing operation by many different means including remotely drawing arrows or other symbols on graph 212 or sending text or voice messages to the mobile device 216.

FIG. 15 is an example of a used anesthesia monitoring graph 222 displayed on a mobile device similar to FIG. 14 but showing the marks, indicated at 224, made by an anesthesia administrator during the course of an operation. Such a graph or facsimile thereof would typically be stored by the hospital or medical facility as a permanent part of the patient's medical records.

Although particular embodiments have been described in this disclosure, many other variations and modifications will be apparent to those skilled in the art. Thus the instant invention can be defined and limited only by the below claims.

What is claimed is:

1. A method for the electronic interpretation of patient vital signs during an operation, comprising:
making a mark corresponding to the symbol of a vital sign on an image of a graph with coordinates displayed on a tablet computer with a touchscreen,
transmitting said mark to a server with a multiplicity of memory locations,
determining the position of said mark relative to said graph coordinates to determine a value for the vital sign, and
recording the value in one of said multiplicity of memory locations of the server.

2. The method of claim 1, wherein handwriting recognition software running on the server is used to interpret the mark made as a vital sign.

3. The method of claim 2, wherein the position of the mark interpreted as a vital sign is used to calculate the value of the vital sign.

4. The method of claim 1, wherein the mark made is unchanged for the duration of the operation.

5. The method of claim 3, wherein the calculated value of the vital sign is automatically compared to a threshold value.

6. The method of claim 1, wherein the marks made are instantaneously transmitted to a supervisor outside the operating room.

7. The method of claim 2, wherein the mark made is interpreted to be a blood pressure symbol.

8. A system for recording patient vital signs during an operation, comprising:
a tablet computer with a touchscreen displaying an image of an anesthesia graph, and
a server with a multiplicity of memory locations in wireless communication with said tablet computer whereupon
a mark corresponding to the symbol of a patient vital sign made on said image of an anesthesia graph is transmitted to the server, whereupon the server interprets said mark relative to said graph coordinates, and then stores the value determined in one of said multiplicity of memory locations.

9. The system of claim 8, wherein the mark made on the tablet computer is unchanged for the duration of the patient's operation.

10. The system of claim 8, wherein the mark made is interpreted by handwriting recognition software running on the server.

11. The system of claim 10, wherein the mark made is interpreted by the handwriting recognition software running on the server to be a blood pressure symbol.

12. The system of claim 8, wherein the value determined is automatically compared to a threshold value.

13. The system of claim 8, wherein the marks made are transmitted in real time to a supervisor outside the operating room.

14. The system of claim 11, wherein blood pressure values are recorded every five minutes.

15. A device for recording the vital signs of a patient undergoing an operation, comprising:
a tablet computer with a touchscreen displaying an image of an anesthesia graph in wireless communication with a server having a multiplicity of memory locations such that a mark corresponding to a symbol for a patient vital sign made on said image of an anesthesia graph is interpreted by said server and stored in one of said multiplicity of memory locations.

16. The device of claim 15 wherein a mark made on the image of the anesthesia graph is unchanged for the duration of the patient's operation.

17. The device of claim 15 wherein handwriting recognition software running on the server is used to interpret marks made as patient vital signs.

18. The device of claim 17 wherein the value of the patient vital sign is calculated by software running on the server using the sign coordinates relative to the anesthesia graph axes.

19. The device of claim 18 wherein the calculated value of the patient vital sign is automatically compared to a threshold value.

20. The device of claim 17 wherein a mark is interpreted to be a blood pressure vital sign.
